(19) United States
(12) Patent Application Publication
(10) Pub. No.: US 2011/0160594 A1
(43) Pub. Date: Jun. 30, 2011

(54) Method for Testing Substances or Substance Mixtures and the Use Thereof

(75) Inventors: Herbert Platsch, Mannheim (DE); Helmut Schiffer, Grossfischlingen (DE); Rolf Hellmann, Lustadt (DE); Stefan Emig, Ludwigshafen (DE); Wolfgang Grabarse, Mannheim (DE); Harald Koehle, Bobenheim (DE); Klaus Grossmann, Neuhofen (DE); Juergen Langewald, Mannheim (DE); Christine Boeddinghaus, Mannheim (DE); John Dorsch, Raleigh, NC (US)

(73) Assignee: BASF SE, Ludwigshafen (DE)

(21) Appl. No.: 13/061,767

(22) PCT Filed: Sep. 4, 2009
(86) PCT No.: PCT/EP09/61469

§ 371 Date: Mar. 2, 2011

Related U.S. Application Data
(60) Provisional application No. 61/094,534, filed on Sep. 5, 2008.

(51) Int. Cl. A61B 6/00; C12Q 1/02

(52) U.S. Cl. 600/473; 435/29

(57) ABSTRACT

The present invention relates to a method for testing a substance or a substance mixture and the use thereof for identifying and/or characterizing the mode of action of the substance or the substance mixture or for determining the biochemical and/or metabolic state of an organism or group of organisms or part(s) thereof after exposure to the substance or the substance mixture.

The present invention relates to a method for testing a substance or a substance mixture and the use of said method in the agrochemical field, in particular for identifying and/or characterizing the mode of action of the substance or the substance mixture or for determining the biochemical and/or metabolic state of an organism or group of organisms or part(s) thereof subsequent to its exposure to the substance or the substance mixture.

WO 03/042406 (US 2005/0123917) describes a method for the characterisation and/or identification of mode of action mechanisms of antimicrobially acting test substances with the aid of IR (infrared), FT-IR (Fourier-Transform infrared), Raman or FT-Raman (Fourier-Transform Raman) analyses. This method comprises a treatment of corresponding microbial cell cultures with the test substance.

When it comes to the characterisation and/or identification of mode of action mechanisms of pesticides the method described in WO 03/042406 is not suitable. Pesticides act on complex organisms and the elucidation of their mode of action requires tests on said organisms.

It is an object of the present invention to provide a practicable method for testing substances or substance mixtures which allows their mode of action on an entire organism to be recognized or the biochemical and/or metabolic state of an orgamism or group of organisms or part(s) thereof be determined subsequent to its exposure to the substance or the substance mixture.

This object is achieved by the present invention through the use of IR, FT-IR, Raman, FT-Raman or Near Infrared (NIR) analyses on organisms previously treated with the test substance or test substance mixture.

The present invention relates to a method for testing a substance or substance mixture, which comprises:
a) exposing an organism or a group of organisms to the substance or to the substance mixture;
b) optionally converting the organism or group of organisms or part(s) thereof into a homogeneous sample;
c) recording at least one spectrum from the group of IR, FT-IR, Raman, FT-Raman and Near Infrared (NIR) on the sample; and
d) comparing the spectrum with one or more reference spectra.

a) Exposure

In step a) of the method, an organism or group of organisms is exposed to the substance to be tested or to the substance mixture to be tested. The aim is to identify the changes in said organism(s) or group of organisms or part(s) thereof associated with the exposure.

According to the present invention, the exposure is carried out in vivo. To this end, the entire organism or group of entire organisms is brought into contact with the substance or substance mixture.

A group of organisms is meant to denote 2 or more individual organisms belonging to the same species. It might be advantageous that the organisms of the group are clones.

According to one particular embodiment, the organism is selected from the group consisting of arthropods, nematodes and molluscs.

Arthropodes Include in Particular:

insects from the order of the lepidopterans (Lepidoptera), for example Agrotis ypsilon, Agrotis segetum, Alabama argillacea, Anticarsia gemmatalis, Argyresthia conjugella, Autographa gamma, Bupalus piniarius, Cacoecia murinana, Capua reticulana, Cheimatobia brumata, Choristoneura fumiferana, Choristoneura occidentalis, Cirphis unipuncta, Cydia pomonella, Dendrolimus pini, Diaphania nitidalis, Diatraea grandiosella, Earias insulana, Elasmopalpus lignosellus, Eupoecilia ambiguella, Evetria bouliana, Feltia subterranea, Galleria mellonella, Grapholitha funebrana, Grapholitha molesta, Heliothis armigera, Heliothis virescens, Heliothis zea, Hellula undalis, Hibernia defoliaria, Hyphantria cunea, Hyponomeuta malinellus, Keiferia lycopersicella, Lambdina fiscellaria, Laphygma exigua, Leucoptera coffeella, Leucoptera scitella, Lithocolletis blancardella, Lobesia botrana, Loxostege sticticalis, Lymantria dispar, Lymantria monacha, Lyonetia clerkella, Malacosoma neustria, Mamestra brassicae, Orgyia pseudotsugata, Ostrinia nubilalis, Panolis flammea, Pectinophora gossypiella, ,Peridroma saucia, Phalera bucephala, Phthorimaea operculella, Phyllocnistis citrella, Pieris brassicae, Plathypena scabra, Plutella xylostella, Pseudoplusia includens, Rhyacionia frustrana, Scrobipalpula absoluta, Sitotroga cerealella, Sparganothis pilleriana, Spodoptera frugiperda, Spodoptera littoralis, Spodoptera litura, Thaumatopoea pityocampa, Tortrix viridana, Trichoplusia ni and Zeiraphera canadensis;

beetles (Coleoptera), for example Agrilus sinuatus, Agriotes lineatus, Agriotes obscurus, Amphimallus solstitialis, Anisandrus dispar, Anthonomus grandis, Anthonomus pomorum, Aphthona euphoridae, Athous haemorrhoidalis, Atomaria linearis, Blastophagus piniperda, Blitophaga undata, Bruchus rufimanus, Bruchus pisorum, Bruchus lentis, Byctiscus betulae, Cassida nebulosa, Cerotoma trifurcata, Cetonia aurata, Ceuthorrhynchus assimilis, Ceuthorrhynchus napi, Chaetocnema tibialis, Conoderus yespertinus, Crioceris asparagi, Ctenicera ssp., Diabrotica longicomis, Diabrotica semipunctata, Diabrotica 12-punctata Diabrotica speciosa, Diabrotica virgifera, Epilachna varivestis, Epitrix hirtipennis, Eutinobothrus brasiliensis, Hylobius abietis, Hypera brunneipennis, Hypera postica, Ips typographus, Lema bilineata, Lema melanopus, Leptinotarsa decemlineata, Limonius californicus, Lissorhoptrus oryzophilus, Melanotus cornmunis, Meligethes aeneus, Melolontha hippocastani, Melolontha melolontha, Oulema oryzae, Ortiorrhynchus sulcatus, Otiorrhynchus ovatus, Phaedon cochleariae, Phyllobius pyri, Phyllotreta chrysocephala, Phyllophaga sp., Phyllopertha horticola, Phyllotreta nemorum, Phyllotreta striolata, Popillia japonica, Sitona lineatus and Sitophilus granaria;

flies, mosquitoes (Diptera), e.g. Aedes aegypti, Aedes albopictus, Aedes vexans, Anastrepha ludens, Anopheles maculipennis, Anopheles crucians, Anopheles albimanus, Anopheles gambiae, Anopheles freeborni, Anopheles leucosphyrus, Anopheles minimus, Anopheles quadrimaculatus, Calliphora vicina, Ceratitis capitata, Chrysomya bezziana, Chrysomya hominivorax, Chrysomya macellaria, Chrysops discalis, Chrysops silacea, Chrysops atlanticus, Cochliomyia hominivorax, Contarinia sorghicola Cordylobia anthropophaga, Culicoides furens, Culex pipiens, Culex nigripalpus, Culex quinquefasciatus, Culex tarsalis, Culiseta inornata, Culiseta melanura, Dacus cucurbitae, Dacus oleae, Dasineura brassicae, Delia antique, Delia coarctata, Delia platura, Delia radicum, Dermatobia hominis, Fannia canicularis, Geomyza Tripunctata, Gasterophilus intestinalis, Glossina morsitans, Glossina palpalis, Glossina fuscipes, Glossina tachinoides, Haematobia irritans, Haplodiplosis equestris, Hippelates spp., Hylemyia platura, Hypoderma lineata, Leptoconops torrens, Liriomyza sativae, Liriomyza trifolii, Lucilia cuprina, Lucilia cuprina, Lucilia sericata, Lycoria pectoralis, Mansonia titiflanus, Mayetiola destructor, Musca domestica, Muscina stabulans, Oestrus ovis, Opomyza florum, Oscinella frit, Pegomya hysocyami, Phorbia antiqua, Phorbia brassicae, Phorbia coarctate, Phlebotomus argentipes, Psorophora columbiae, Psila rosae, Psorophora discolor, Prosimulium mixtum, Rhagoletis cerasi, Rhagoletis pomonella, Sarcophaga haemorrhoidalis, Sarcophaga sp., Simulium vittatum, Stomoxys calcitrans, Tabanus bovinus, Tabanus atratus, Tabanus lineola, and Tabanus similis, Tipula oleracea, and Tipula paludosa;

thrips (Thysanoptera), e.g. Dichromothrips corbetti, Dichromothrips ssp, Frankliniella fusca, Frankliniella occidentalis, Frankliniella tritici, Scirtothrips citri, Thrips oryzae, Thrips palmi and Thrips tabaci;

termites (Isoptera), e.g. Calotermes flavicollis, Leucotermes flavipes, Heterotermes aureus, Reticulitermes flavipes, Reticulitermes virginicus, Reticulitermes lucifugus, Termes natalensis, and Coptotermes formosanus;

cockroaches (Blattaria-Blattodea), e.g. Blattella germanica, Blattella asahinae, Periplaneta americana, Periplaneta japonica, Periplaneta brunnea, Periplaneta fuligginosa, Periplaneta australasiae, and Blatta orientalis;

true bugs (Hemiptera), e.g. Acrostemum hilare, Blissus leucopterus, Cyrtopeltis notatus, Dysdercus cingulatus, Dysdercus intermedius, Eurygaster integriceps, Euschistus impictiventris, Leptoglossus phyllopus, Lygus lineolaris, Lygus pratensis, Nezara viridula, Piesma quadrata, Solubea insularis, Thyanta perditor, Acyrthosiphon onobrychis, Adelges laricis, Aphidula nasturtii, Aphis fabae, Aphis forbesi, Aphis pomi, Aphis gossypii, Aphis grossu/ariae, Aphis schneideri, Aphis spiraecola, Aphis sambuci, Acyrthosiphon pisum, Aulacorthum solani, Bemisia argentifolii, Brachycaudus cardui, Brachycaudus helichrysi, Brachycaudus persicae, Brachycaudus prunicola, Brevicoryne brassicae, Capitophorus horni, Cerosipha gossypii, Chaetosiphon fragaefolii, Cryptomyzus ribis, Dreyfusia nordmannianae, Dreyfusia piceae, Dysaphis radicola, Dysaulacorthum pseudosolani, Dysaphis plantaginea, Dysaphis pyri, Empoasca fabae, Hyalopterus pruni, Hyperomyzus Iactucae, Macrosiphum avenae, Macrosiphum euphorbiae, Macrosiphon rosae, Megoura viciae, Melanaphis pyrarius, Metopolophium dirhodum, Myzus persicae, Myzus ascalonicus, Myzus cerasi, Myzus varians, Nasonovia ribis-nigri, Nilaparvata lugens, Pemphigus bursarius, Perkinsiella saccharicida, Phorodon humuli, Psylla mali, Psylla piri, Rhopalomyzus ascalonicus, Rhopalosiphum maidis, Rhopalosiphum padi, Rhopalosiphum insertum, Sappaphis mala, Sappaphis mali, Schizaphis graminum, Schizoneura lanuginosa, Sitobion avenae, Trialeurodes vaporariorum, Toxoptera aurantiiand, Viteus vitifolii, Cimex lectularius, Cimex hemipterus, Reduvius senilis, Triatoma spp., and Arilus critatus;

ants, bees, wasps, sawflies (Hymenoptera), e.g. Athalia rosae, Atta cephalotes, Atta capiguara, Atta cephalotes, Atta laevigata, Atta robusta, Atta sexdens, Atta texana, Crematogaster spp., Hoplocampa minuta, Hoplocampa testudinea, Monomorium pharaonis, Solenopsis geminata, Solenopsis invicta, Solenopsis richteri, Solenopsis xyloni, Pogonomyrmex barbatus, Pogonomyrmex californicus, Pheidole megacephala, Dasymutilla occidentalis, Bombus spp. Vespula squamosa, Paravespula vulgaris, Paravespula pennsylvanica, Paravespula germanica, Dolichovespula maculata, Vespa crabro, Polistes rubiginosa, Camponotus floridanus, and Linepithema humile;

crickets, grasshoppers, locusts (Orthoptera), e.g. Acheta domestica, Gryllotalpa gryllotalpa, Locusta migratoria, Melanoplus bivittatus, Melanoplus femurrubrum, Melanoplus mexicanus, Melanoplus sanguinipes, Melanoplus spretus, Nomadacris septemfasciata, Schistocerca americana, Schistocerca gregaria, Dociostaurus maroccanus, Tachycines asynamorus, Oedaleus senegalensis, Zonozerus variegatus, Hieroglyphus daganensis, Kraussaria angulifera, Calliptamus italicus, Chortoicetes terminifera, and Locustana pardalina;

Arachnoidea, such as arachnids (Acarina), e.g. of the families Argasidae, Ixodidae and Sarcoptidae, such as Amblyomma americanum, Amblyomma variegatum, Ambryomma maculatum, Argas persicus, Boophilus annulatus, Boophilus decoloratus, Boophilus microplus, Dermacentor silvarum, Dermacentor andersoni, Dermacentor variabilis, Hyalomma truncatum, Ixodes ricinus, Ixodes rubicundus, Ixodes scapularis, Ixodes holocyclus, Ixodes pacificus, Ornithodorus moubata, Ornithodorus hermsi, Ornithodorus turicata, Ornithonyssus bacoti, Otobius megnini, Dermanyssus gallinae, Psoroptes ovis, Rhipicephalus sanguineus, Rhipicephalus appendiculatus, Rhipicephalus evertsi, Sarcoptes scabiei, and Eriophyidae spp. such as Aculus schlechtendali, Phyllocoptrata oleivora and Eriophyes sheldoni; Tarsonemidae spp. such as Phytonemus pallidus and Polyphagotarsonemus latus; Tenuipalpidae spp. such as Brevipalpus phoenicis; Tetranychidae spp. such as Tetranychus cinnabarinus, Tetranychus kanzawai, Tetranychus pacificus, Tetranychus telarius and Tetranychus urticae, Panonychus ulmi, Panonychus citri, and Oligonychus pratensis; Araneida, e.g. Latrodectus mactans, and Loxosceles reclusa;

fleas (Siphonaptera), e.g. Ctenocephalides fells, Ctenocephalides canis, Xenopsylla cheopis, Pulex irritans, Tunga penetrans, and Nosopsyllus fasciatus;

silverfish, firebrat (Thysanura), e.g. Lepisma saccharina and Thermobia domestica;

centipedes (Chilopoda), e.g. Scutigera coleoptrata;

millipedes (Diplopoda), e.g. Narceus spp.;

earwigs (Dermaptera), e.g. forficula auricularia; and

lice (Phthiraptera), e.g. Pediculus humanus capitis, Pediculus humanus corporis, Pthirus pubis, Haematopinus eurystemus, Haematopinus suis, Linognathus vituli, Bovicola bovis, Menopon gallinae, Menacanthus stramineus and Solenopotes capillatus.

Particular preference is given to Megoura viciae, Myzus persicae, Aphis fabae, Aphis gossypii, Aedes aegypti, Drosophila melanogaster, Spodoptera frugiperda, Spodoptera littoralis, and Spodoptera litura.

Nematodes include in particular root-knot nematodes, e.g. Meloidogyne arenaria, Meloidogyne chitwoodi, Meloidogyne exigua, Meloidogyne hapla, Meloidogyne incognita, Meloidogyne javanica and other Meloidogyne species; cyst nematodes, e.g. Globodera rostochiensis, Globodera pallida, Globodera tabacum and other Globodera species, Heterodera avenae, Heterodera glycines, Heterodera schachtii, Heterodera trifolii, and other Heterodera species; seed gall nematodes, e.g. Anguina funesta, Anguina tritici and other Anguina species; stem and foliar nematodes, e.g. Aphelenchoides besseyi, Aphelenchoides fragariae, Aphelenchoides ritzemabosi and other Aphelenchoides species; sting nematodes, e.g. Belonolaimus longicaudatus and other Belonolaimus species; pine nematodes, e.g. Bursaphelenchus xylophilus and other Bursaphelenchus species; ring nematodes, e.g. Criconema species, Criconemella species, Criconemoides species, and Mesocriconema species; stem and bulb nematodes, e.g. Ditylenchus destructor, Ditylenchus dipsaci, Ditylenchus myceliophagus and other Ditylenchus species; awl nematodes, e.g. Dolichodorus species; spiral nematodes, Helicotylenchus dihystera, Helicotylenchus multicinctus and other Helicotylenchus species, Rotylenchus robustus and other Rotylenchus species; sheath nematodes, e.g. Hemicycliophora species and Hemicriconemoides species; Hirshmanniella species; lance nematodes, e.g. Hoplolaimus columbus, Hoplolaimus galeatus and other Hoplolaimus species; false root-knot nematodes, e.g. Nacobbus aberrans and other Nacobbus species; needle nematodes, e.g. Longidorus elongates and other Longidorus species; pin nematodes, e.g. Paratylenchus species; lesion nematodes, e.g. Pratylenchus brachyurus, Pratylenchus coffeae, Pratylenchus curvitatus, Pratylenchus goodeyi, Pratylencus neglectus, Pratylenchus penetrans, Pratylenchus scribneri, Pratylenchus vulnus, Pratylenchus zeae and other Pratylenchus species; Radinaphelenchus cocophilus and other Radinaphelenchus species; burrowing nematodes, e.g. Radopholus similis and other Radopholus species; reniform nematodes, e.g. Rotylenchulus reniformis and other Rotylenchulus species; Scutellonema species; stubby root nematodes, e.g. Trichodorus primitivus and other Trichodorus species; Paratrichodorus minor and other Paratrichodorus species; stunt nematodes, e.g. Tylenchorhynchus claytoni, Tylenchorhynchus dubius and other Tylenchorhynchus species and Merlinius species; citrus nematodes, e.g. Tylenchulus semipenetrans and other Tylenchulus species; dagger nematodes, e.g. Xiphinema americanum, Xiphinema index, Xiphinema diversicaudatum and other Xiphinema species; and other nematode species such as Caenorhabditis elegans.

Particular preference is given to Meloidogyne exigua and Caenorhabditis elegans.

Molluscs include in particular terrestrial and amphibious snails and slugs, for example those of the genera Deroceras (Agriolimax), Arianta, Limax, Helix, Helicogona, Cepaea, Milax, Lymnaea (Galba), Achatina, Theba, Cochlicella, Helicarion and Vaginulus. The snail and slug pests include, for example, the slugs Anion ater, A. lusitanicus, A. hortensis, Agriolimax reticulatus, Limax flavus, L. maximus, Milax gagates, Mariaella dursumierei, Helicarion salius, Vaginula hedleyi and Pamarion pupillaris and the snails Helix aspersa spp., Cepaea nemoralis, Theba pisana, Achatina fulica, A. zanzibarica, Limicolaria kambeul, Bradybaena spp., Cochlodina spp., Helicella spp., Euomphalia spp. and Arianta arbustorum.

According to a further particular embodiment, the organism is a plant.

As used herein, the term “plant” means an entire plant, be it genetically modified or not. The term “entire plant” refers to a complete plant individual in its vegetative, i.e. non-seed stage, characterized by the presence of an arrangement of roots, shoots and foliage, depending on the developmental stage of the plant also flowers and/or fruits, all of which are physically connected to form an individual which is, under reasonable conditions, viable without the need for artificial measures.

The term “plant” may also refer to seed. As used herein, the term “seed” denotes any resting stage of a plant that is physically detached from the vegetative stage of a plant and/or may be stored for prolonged periods of time and/or can be used to re-grow another plant individual of the same species. Here, the term “resting” refers to a state wherein the plant retains viability, within reasonable limits, in spite of the absence of light, water and/or nutrients essential for the vegetative (i.e. non-seed) state. In particular, the term refers to true seeds but does not embraces plant propagules such as suckers, corms, bulbs, fruit, tubers, grains, cuttings and cut shoots.

Suitable plants include, but are not limited to, the following:

Monocotyledonous weeds, in particular annual weeds such as gramineous weeds (grasses) including Echinochloa species such as barnyardgrass (Echinochloa crusgalli var. crus-galli), Digitaria species such as crabgrass (Digitaria sanguinalis), Setaria species such as green foxtail (Setaria viridis) and giant foxtail (Setaria faberii), Sorghum species such as johnsongrass (Sorghum halepense Pers.), Avena species such as wild oats (Avena fatua), Cenchrus species such as Cenchrus echinatus, Bromus species, Lolium species, Phalaris species, Eriochloa species, Panicum species, Brachiaria species, annual bluegrass (Poa annua), blackgrass (Alopecurus myosuroides), Aegilops cylindrica, Agropyron repens, Apera spica-venti, Eleusine indica, Cynodon dactylon and the like.

Dicotyledonous weeds, in particular broad leaf weeds including Polygonum species such as wild buckwheat (Polygonum convolvolus), Amaranthus species such as pigweed (Amaranthus retroflexus), Chenopodium species such as common lambsquarters (Chenopodium album L.), Sida species such as prickly sida (Sida spinosa L.), Ambrosia species such as common ragweed (Ambrosia artemisiifolia), Acanthospermum species, Anthemis species, Atriplex species, Cirsium species, Convolvulus species, Conyza species, Cassia species, Commelina species, Datura species, Euphorbia species, Geranium species, Galinsoga species, morningglory (Ipomoea species), Lamium species, Malva species, Matricaria species, Sysimbrium species, Solanum species, Xanthium species, Veronica species, Viola species, common chickweed (Stellaria media), velvetleaf (Abutilon theophrasti), Hemp sesbania (Sesbania exaltata Cory), Anoda cristata, Bidens pilosa, Brassica kaber, Capsella bursa-pastoris, Centaurea cyanus, Galeopsis tetrahit, Galium aparine, Helianthus annuus, Desmodium tortuosum, Kochia scoparia, Mercurialis annua, Myosotis arvensis, Papaver rhoeas, Raphanus raphanistrum, Salsola kali, Sinapis arvensis, Sonchus arvensis, Thlaspi arvense, Tagetes minuta, Richardia brasiliensis, and the like.

Annual and perennial sedge weeds including cyperus species such as purple nutsedge (Cyperus rotundus L.), yellow nutsedge (Cyperus esculentus L.), hime-kugu (Cyperus brevifolius H.), sedge weed (Cyperus microiria Steud), rice flatsedge (Cyperus iria L.), and the like.

Suitable plants can also be
grain crops, including e.g.
   cereals such as wheat (Triticum aestivum) and wheat like crops such as durum (T. durum), einkorn (T. monococcum), emmer (T. dicoccon) and spelt (T. spelta), rye (Secale cereale), triticale (Tritiosecale), barley (Hordeum vulgare);
   maize (corn; Zea mays);
   sorghum (e.g. Sorghum bicolour);
   rice (Oryza spp. such as Oryza sativa and Oryza glaberrima); and
   sugar cane;
legumes (Fabaceae), including e.g. soybeans (Glycine max.), peanuts (Arachis hypogaea and pulse crops such as peas including Pisum sativum, pigeon pea and cowpea, beans including broad beans (Vicia faba), Vigna spp., and Phaseolus spp. and lentils (lens culinaris var.);
brassicaceae, including e.g. canola (Brassica napus), oilseed rape (Brassica napus), cabbage (B. oleracea var.), mustard such as B. juncea, B. campestris, B. narinosa, B. nigra and B. tournefortii; and turnip (Brassica rapa var.);
other broadleaf crops including e.g. sunflower, cotton, flax, linseed, sugarbeet, potato and tomato;
TNV-crops (TNV: trees, nuts and vine) including e.g. grapes, citrus, pomefruit, e.g. apple and pear, coffee, pistachio and oilpalm, stonefruit, e.g. peach, almond, walnut, olive, cherry, plum and apricot; turf, pasture and rangeland;
onion and garlic;
bulb ornamentals such as tulips and narcissus;
conifers and deciduous trees such as pinus, fir, oak, maple, dogwood, hawthorne, crabapple, and rhamnus (buckthorn); and
garden ornamentals such as petunia, marigold, roses and snapdragon.

According to one particular embodiment, the organism is selected from the group consisting of wheat, barley, rye, triticale, durum, rice, corn, sugarcane, sorghum, soybean, pulse crops such as pea, bean and lentils, peanut, sunflower, sugarbeet, potato, cotton, brassica crops, such as oilseed rape, canola, mustard, cabbage and turnip, turf, grapes, pomefruit, such as apple and pear, stonefruit, such as peach, almond, walnut, olive, cherry, plum and apricot, citrus, coffee, pistachio, garden ornamentals, such as roses, petunia, marigold, snap dragon, bulb ornamentals such as tulips and narcissus, onion, garlic, conifers and deciduous trees such as pinus, fir, oak, maple, dogwood, hawthorne, crabapple and rhamnus.

Suitable plants can also be crop plants which are resistant to one or more herbicides owing to genetic engineering or breeding, which are resistant to one or more pathogens such as plant pathogenous fungi owing to genetic engineering or breeding, or which are resistant to attack by insects owing to genetic engineering or breeding.

According to a further particular embodiment stress resistant plants including genetically modified plants were used as selected organisms.

Of particular importance are also model plants used in physiological and biotechnological research including Brachypodium distachyon (Poaceae), Populus trichocarpa (Salicaceae), Physcomitrella patens (Bryophyta), Medicago truncatula (Fabaceae) and small plants of the plant families of Brassicaceae (including Arabidopsis species) and Lemnaceae (including Lemna species).

According to a further particular embodiment, the organism is selected from the family of Lemnaceae consisting of different subfamilies, genera, sections and species. Subfamily Lemnoideae includes genera Spirodela with section Spirodela and species S. intermedia, S. polyrrhiza, and section Oligorrhizae and species S. punctata; genera Lemna with section Lemna and species L. gibba, L. disperma, L. minor, L. japonica, L. obscura, L. ecuadoriensis, L. turionifera, L. paucicostata, section Hydrophylla and species L. trisulca, section Alatae and species L. perpusilla, L. aequinoctialis, section Biformes and species L. tenera, section Uninerves and species L. valdiviana, L. minuscula. Subfamily Wolffioideae includes genera Wolffiella with section Stipitatae and species W. hyalina, W. repanda, section Rotundae and species W. rotunda, section Wolffiella and species W. neotropica, W. Welwitschii, W. lingulata, W. oblonga, W. gladiata, W. denticulata; genera Wolffia with section Pseudorrhizae and species W. microscopia, section Elongatae and species W. elongata, section Pigmentatae and species W. brasiliensis, W. borealis, section Wolffia and species W. australiana, W. angusta, W. arrhiza, W. columbiana, W. globosa.

According to a further particular embodiment, the organism is a fungus.

Suitable fungi include, but are limited to, the following:

Albugo spp. (white rust) on ornamentals, vegetables (e. g. A. candida) and sunflowers (e. g. A. tragopogonis); Alternaria spp. (Alternaria leaf spot) on vegetables, rape (A. brassicola or brassicae), sugar beets (A. tenuis), fruits, rice, soybeans, potatoes (e. g. A. solani or A. alternata), tomatoes (e. g. A. solani or A. alternata) and wheat; Aphanomyces spp. on sugar beets and vegetables; Ascochyta spp. on cereals and vegetables, e. g. A. tritici (anthracnose) on wheat and A. hordei on barley; Bipolaris and Drechslera spp. (teleomorph: Cochliobolus spp.) on corn (e. g. D. maydis), cereals (e. g. B. sorokiniana: spot blotch), rice (e. g. B. oryzae) and turfs; Blumeria (formerly Erysiphe) graminis (powdery mildew) on cereals (e. g. on wheat or barley); Botrytis cinerea (teleomorph: Botryotinia fuckeliana: grey mold) on fruits and berries (e. g. strawberries), vegetables (e. g. lettuce, carrots, celery and cabbages), rape, flowers, vines, forestry plants and wheat; Bremia lactucae (downy mildew) on lettuce; Ceratocystis (syn. Ophiostoma) spp. (rot or wilt) on broad-leaved trees and evergreens, e. g. C. ulmi (Dutch elm disease) on elms; Cercospora spp. (Cercospora leaf spots) on corn, rice, sugar beets (e. g. C. beticola), sugar cane, vegetables, coffee, soybeans (e. g. C. sojina or C. kikuchii) and rice; Cladosporium spp. on tomatoes (e. g. C. fulvum: leaf mold) and cereals, e. g. C. herbarum (black ear) on wheat; Claviceps purpurea (ergot) on cereals; Cochliobolus (anamorph: Helminthosporium of Bipolaris) spp. (leaf spots) on corn (C. carbonum), cereals (e. g. C. sativus, anamorph: B. sorokiniana) and rice (e. g. C. miyabeanus, anamorph: H. oryzae); Colletotrichum (teleomorph: Glomerella) spp. (anthracnose) on cotton (e. g. C. gossypii), corn (e. g. C. graminicola), soft fruits, potatoes (e. g. C. coccodes: black dot), beans (e. g. C. lindemuthianum) and soybeans (e. g. C. truncatum or C. gloeosporioides); Corticium spp., e. g. C. sasakii (sheath blight) on rice; Corynespora cassiicola (leaf spots) on soybeans and ornamentals; Cycloconium spp., e. g. C. oleaginum on olive trees; Cylindrocarpon spp. (e. g. fruit tree canker or young vine decline, teleomorph: Nectria or Neonectria spp.) on fruit trees, vines (e. g. C. liriodendri, teleomorph: Neonectria liriodendri: Black Foot Disease) and ornamentals; Dematophora (teleomorph: Rosellinia) necatrix (root and stem rot) on soybeans; Diaporthe spp., e. g. D. phaseolorum (damping off) on soybeans; Drechslera (syn. Helminthosporium, teleomorph: Pyrenophora) spp. on corn, cereals, such as barley (e. g. D. teres, net blotch) and wheat (e. g. D. tritici-repentis: tan spot), rice and turf; Esca (dieback, apoplexy) on vines, caused by Formitiporia (syn. Phellinus) punctata, F. mediterranea, Phaeomoniella chlamydospora (earlier Phaeoacremonium chlamydosporum), Phaeoacremonium aleophilum and/or Botryosphaeria obtuse; Elsinoe spp. on pome fruits (E. pyri), soft fruits (E. veneta: anthracnose) and vines (E. ampelina: anthracnose); Entyloma oryzae (leaf smut) on rice; Epicoccum spp. (black mold) on wheat; Erysiphe spp. (powdery mildew) on sugar beets (E. betae), vegetables (e. g. E. pisi), such as cucurbits (e. g. E. cichoracearum), cabbages, rape (e. g. E. cruciferarum); Eutypa lata (Eutypa canker or dieback, anamorph: Cytosporina lata, syn. Libertella blepharis) on fruit trees, vines and ornamental woods; Exserohilum (syn. Helminthosporium) spp. on corn (e. g. E. turcicum); Fusarium (teleomorph: Gibberella) spp. (wilt, root or stem rot) on various plants, such as F. graminearum or F. culmorum (root rot, scab or head blight) on cereals (e. g. wheat or barley), F. oxysporum on tomatoes, F. solani on soybeans and F. verticillioides on corn; Gaeumannomyces graminis (take-all) on cereals (e. g. wheat or barley) and corn; Gibberella spp. on cereals (e. g. G. zeae) and rice (e. g. G. fujikuroi: Bakanae disease); Glomerella cingulata on vines, pome fruits and other plants and G. gossypii on cotton; Grainstaining complex on rice; Guignardia bidwellii (black rot) on vines; Gymnosporangium spp. on rosaceous plants and junipers, e. g. G. sabinae (rust) on pears; Helminthosporium spp. (syn. Drechslera, teleomorph: Cochliobolus) on corn, cereals and rice; Hemileia spp., e. g. H. vastatrix (coffee leaf rust) on coffee; lsariopsis clavispora (syn. Cladosporium vitis) on vines; Macrophomina phaseolina (syn. phaseoli) (root and stem rot) on soybeans and cotton; Microdochium (syn. Fusarium) nivale (pink snow mold) on cereals (e. g. wheat or barley); Microsphaera diffusa (powdery mildew) on soybeans; Monilinia spp., e. g. M. laxa, M. fructicola and M. fructigena (bloom and twig blight, brown rot) on stone fruits and other rosaceous plants; Mycosphaerella spp. on cereals, bananas, soft fruits and ground nuts, such as e. g. M. graminicola (anamorph: Septoria tritici, Septoria blotch) on wheat or M. fijiensis (black Sigatoka disease) on bananas; Peronospora spp. (downy mildew) on cabbage (e. g. P. brassicae), rape (e. g. P. parasitica), onions (e. g. P. destructor), tobacco (P. tabacina) and soybeans (e. g. P. manshurica); Phakopsora pachyrhizi and P. meibomiae (soybean rust) on soybeans; Phialophora spp. e. g. on vines (e. g. P. tracheiphila and P. tetraspora) and soybeans (e. g. P. gregata: stem rot); Phoma lingam (root and stem rot) on rape and cabbage and P. betae (root rot, leaf spot and damping-off) on sugar beets; Phomopsis spp. on sunflowers, vines (e. g. P. viticola: can and leaf spot) and soybeans (e. g. stem rot: P. phaseoli, teleomorph: Diaporthe phaseolorum); Physoderma maydis (brown spots) on corn; Phytophthora spp. (wilt, root, leaf, fruit and stem root) on various plants, such as paprika and cucurbits (e. g. P. capsici), soybeans (e. g. P. megasperma, syn. P. sojae), potatoes and tomatoes (e. g. P. infestans: late blight) and broad-leaved trees (e. g. P. ramorum: sudden oak death); Plasmodiophora brassicae (club root) on cabbage, rape, radish and other plants; Plasmopara spp., e. g. P. viticola (grapevine downy mildew) on vines and P. halstedii on sunflowers; Podosphaera spp. (powdery mildew) on rosaceous plants, hop, pome and soft fruits, e. g. P. leucotricha on apples; Polymyxa spp., e. g. on cereals, such as barley and wheat (P. graminis) and sugar beets (P. betae) and thereby transmitted viral diseases; Pseudocercosporella herpotrichoides (eyespot, teleomorph: Tapesia yallundae) on cereals, e. g. wheat or barley; Pseudoperonospora (downy mildew) on various plants, e. g. P. cubensis on cucurbits or P. humili on hop; Pseudopezicula tracheiphila (red fire disease or ‘rotbrenner’, anamorph: Phialophora) on vines; Puccinia spp. (rusts) on various plants, e. g. P. triticina (brown or leaf rust), P. striiformis (stripe or yellow rust), P. hordei (dwarf rust), P. graminis (stem or black rust) or P. recondita (brown or leaf rust) on cereals, such as e. g. wheat, barley or rye, and asparagus (e. g. P. asparagi); Pyrenophora (anamorph: Drechslera) tritici-repentis (tan spot) on wheat or P. teres (net blotch) on barley; Pyricularia spp., e. g. P. oryzae (teleomorph: Magnaporthe grisea, rice blast) on rice and P. grisea on turf and cereals; Pythium spp. (damping-off) on turf, rice, corn, wheat, cotton, rape, sunflowers, soybeans, sugar beets, vegetables and various other plants (e. g. P. ultimum or P. aphanidermatum); Ramularia spp., e. g. R. collo-cygni (Ramularia leaf spots, Physiological leaf spots) on barley and R. beticola on sugar beets; Rhizoctonia spp. on cotton, rice, potatoes, turf, corn, rape, potatoes, sugar beets, vegetables and various other plants, e. g. R. solani (root and stem rot) on soybeans, R. solani (sheath blight) on rice or R. cerealis (Rhizoctonia spring blight) on wheat or barley; Rhizopus stolonifer (black mold, soft rot) on strawberries, carrots, cabbage, vines and tomatoes; Rhynchosporium secalis (scald) on barley, rye and triticale; Sarocladium oryzae and S. attenuatum (sheath rot) on rice; Sclerotinia spp. (stem rot or white mold) on vegetables and field crops, such as rape, sunflowers (e. g. S. sclerotiorum) and soybeans (e. g. S. rolfsii or S. sclerotiorum); Septoria spp. on various plants, e. g. S. glycines (brown spot) on soybeans, S. tritici (Septoria blotch) on wheat and S. (syn. Stagonospora) nodorum (Stagonospora blotch) on cereals; Uncinula (syn. Erysiphe) necator (powdery mildew, anamorph: Oidium tuckeri) on vines; Setospaeria spp. (leaf blight) on corn (e. g. S. turcicum, syn. Helminthosporium turcicum) and turf; Sphacelotheca spp. (smut) on corn, (e. g. S. reiliana: head smut), sorghum and sugar cane; Sphaerotheca fuliginea (powdery mildew) on cucurbits; Spongospora subterranea (powdery scab) on potatoes and thereby transmitted viral diseases; Stagonospora spp. on cereals, e. g. S. nodorum (Stagonospora blotch, teleomorph: Leptosphaeria [syn. Phaeosphaeria] nodorum) on wheat; Synchytrium endobioticum on potatoes (potato wart disease); Taphrina spp., e. g. T. deformans (leaf curl disease) on peaches and T. pruni (plum pocket) on plums; Thielaviopsis spp. (black root rot) on tobacco, pome fruits, vegetables, soybeans and cotton, e. g. T. basicola (syn. Chalara elegans); Tilletia spp. (common bunt or stinking smut) on cereals, such as e. g. T. tritici (syn. T. caries, wheat bunt) and T. controversa (dwarf bunt) on wheat; Typhula incarnata (grey snow mold) on barley or wheat; Urocystis spp., e. g. U. occulta (stem smut) on rye; Uromyces spp. (rust) on vegetables, such as beans (e. g. U. appendiculatus, syn. U. phaseoli) and sugar beets (e. g. U. betae); Ustilago spp. (loose smut) on cereals (e. g. U. nuda and U. avaenae), corn (e. g. U. maydis: corn smut) and sugar cane; Venturia spp. (scab) on apples (e. g. V. inaequalis) and pears; and Verticillium spp. (wilt) on various plants, such as fruits and ornamentals, vines, soft fruits, vegetables and field crops, e. g. V. dahliae on strawberries, rape, potatoes and tomatoes.

harmful fungi in the protection of materials (e. g. wood, paper, paint dispersions, fiber or fabrics) and in the protection of stored products. As to the protection of wood and construction materials, the particular attention is paid to the following harmful fungi: Ascomycetes such as Ophiostoma spp., Ceratocystis spp., Aureobasidium pullulans, Sclerophoma spp., Chaetomium spp., Humicola spp., Petriella spp., Trichurus spp.; Basidiomycetes such as Coniophora spp., Coriolus spp., Gloeophyllum spp., Lentinus spp., Pleurotus spp., Poria spp., Serpula spp. and Tyromyces spp., Deuteromycetes such as Aspergillus spp., Cladosporium spp., Penicillium spp., Trichorma spp., Alternaria spp., Paecilomyces spp. and Zygomycetes such as Mucor spp., and in addition in the protection of stored products the following yeast fungi are worthy of note: Candida spp. and Saccharomyces cerevisae.

Preferred fungi are Pyricularia oryzae, Septoria tritici, Botrytis cinera, Phytophthora infestans, and Pytium spp.

The substance to be tested or the substance mixture to be tested can be administered to the organism, or brought otherwise into contact with the organism, e.g. by ingestion of feed treated with the substance or substance mixture.

The concentration of test substance or test substance mixture to which the organism or group of organisms is exposed (with which it is brought into contact) should be sufficiently high so as to exert the effect to which a mode of action is to be allocated. Typical concentrations for herbicides, fungicides and insecticides are in the range of 10⁻³ M to 10⁻¹⁰ M.

According to a particular embodiment of the present invention, the substance or the substance mixture to be tested is a pesiticide. Pesticides comprise in particular avicides, acaricides, desiccants, bactericides, chemosterilants, defoliants, antifeedants, fungicides, herbicides, herbicide safeners, insect attractants, insecticides, insect repellents, molluscicides, nematicides, mating disrupters, plant activators, plant growth regulators, rodenticides, mammal repellents, synergists, bird repellents and virucides. According to a particular embodiment, the test substance or test substance mixture is selected from herbicides, insecticides, fungicides, acaricides, nematodicides, plant growth regulators and substances which improve the health of a plant.

According to one embodiment, the test substance or test substance mixture has an insecticidal effect.

As used herein, the terms “insecticidal effect” and “insecticidal activity” mean any direct or indirect action on the target pest (the organism), which may be any arthropod pest and preferably an insect pest, in particular those as listed above including, but not limited to, killing the pest, repelling the pest from the plant seeds, fruits, roots, shoots and/or foliage, inhibiting the feeding of the pest on, or the laying of its eggs on, the plant seeds, fruits, roots, shoots and/or foliage, and inhibiting or preventing reproduction of the pest.

According to another embodiment, the test substance or test substance mixture has an fungicidal effect.

As used herein, the terms “fungicidal effect” and “fungicidal activity” mean any direct or indirect action on the target pest (the organism), which may be any phytopathogenic fungus.

According to another embodiment, the test substance or test substance mixture has an herbicidal effect.

As used herein, the terms “herbicidal effect” and “herbicidal activity” mean any direct or indirect action on the target pest (the organism).

The exposure time may in fact vary. However, firstly a minimum exposure time is important for the method of the invention, so that the effects caused by the exposure can be determined. Secondly, a relatively short exposure time is expedient so that the method can be carried out quickly. Thus, the time may range from a few hours to several days or even several weeks. However, in all indications preference is given according to the invention firstly to a minimum exposure time in the region of more than a few hours, and secondly to a maximum exposure time of up to several days. 24 to 72 hours, e.g. 48 hours, haven proven suitable.

The combination, preferred according to the invention, of in vivo exposure and relatively short exposure times is associated in particular with the following advantages:
the possibility of being able to employ relatively small amounts of substance;
a short time for carrying out the method;
the use of a relatively small number of organisms.

The method of the invention normally comprises choosing in a plurality of organisms different exposure times in order in this way to be able to recognize an exposure time-dependent change in the organism. An analogous statement applies to the dosage (concentration) of the substance to be tested or of the substance mixture to be tested.

It is advantageous according to the invention to record the spectra after at least two exposure times. Repetition of the recording after different exposure times makes it possible to determine the relative change in said organism as a function of time. Such a time-dependent determination permits a significant change over time to be detected as a trend, with the aid of suitable statistical methods, thus allowing transient phenomena to be recognized and evaluated appropriately.

Accordingly, in an advantageous embodiment, the method of the invention comprises
a1) exposing a first organism or first group of organisms to the substance or to the substance mixture;
b1) converting the first organism or first group of organisms into a first homogenized sample;
c1) recording at least one spectrum from the group of IR, FT-IR, Raman, FT-Raman and Near Infrared (NIR) on said first homogenized sample;
a2) exposing a second organism or a second group of organisms to the substance or to the substance mixture; and
b2) converting the second organism or second group of organisms into a second homogenized sample;
c2) recording at least one spectrum from the group of IR, FT-IR, Raman, FT-Raman and Near Infrared (NIR) on said second homogenized sample,
   where the exposure time of the first organism or first group of organisms is different from the exposure time of the second organism or second group of organisms.

The exposure is followed by the treated organism(s), or representative parts of the treated organism being provided as sample, usually with suitable working up, for analytical determination.

b) Sample Preparation

The organism or group of organisms after having been exposed to the test substance or test substance mixture represents the bulk sample. For some bulk samples it will be possible to record the spectra directly. Because of the size of the organism used it is often impractical to use the organism as such for recording the spectrum. Therefore the method of the invention comprises a step wherein the organism (bulk sample) is converted to a form of sample that is accessible for recording the spectra. To this end, it is possible in principle to use any samples derived from the organism, provided the samples reflects the organism's biochemical and/or metabolic state of interest. Thus, the entire organism or group of entire organisms, or a part of the entire organism or group of parts of the entire organisms may be converted to a form of sample that is accessible for recording the spectra. A suitable part of an organism may be, for instance, a body part, an organ or a tissue derived from the organism, such as a part of the body of an insect, e.g. the head, an insect organ, e.g. the brain, an insect tissue, e.g. brain tissue, a part of a plant, e.g. root, shoot, foliage, flower or other parts of the vegetative stage of the plant, a plant organ, a plant tissue, or a plant propagule.

With a view to the spectra to be recorded according to the invention, the organism thus often undergoes a preparative working up, thus converting the organism or part(s) thereof into an expedient form that is suitable for the method of the invention. Such a working up ordinarily corresponds to conventional practice and is based in particular on the requirements of analytical determination and especially of IR, FT-IR, Raman, FT-Raman and Near Infrared (NIR) analysis.

The requirements for suitable sample preparation are usually strict. Artifactual alterations in the sample composition, for example through proteolysis or other modifications (e.g. oxidation, evaporation), should be avoided.

As the organism or part of organism is considered to represent a multi-component material, the preparation of a sample that can be subjected to the spectroscopic analysis usually requires that the organism is initially homogenized. A sample is sufficiently homogeneous if the spectrum recorded on the homogeneous sample is a representative spectrum of the organism(s) or the part(s) thereof from which it is derived.

According to one embodiment, homogenizing comprises processing the organism(s) or part(s) thereof to a powder. This usually requires subjecting the organism(s) or part(s) thereof to size reduction. Size reduction, also known as comminution, is defined as the breakdown of matrices, in particular solids, into smaller particles. Expediently, size reduction is carried out mechanically.

According to a particular embodiment, size reduction according to the present invention is for producing a desired particle size.

Usually, size reduction comprises a step of milling (or grinding or powdering) the organism(s) or part(s) thereof to yield the powder. This step can be performed using any device known in the art to produce the powder and especially a powder having the desired particle size. According to a preferred embodiment, milling is bead milling. For instance, the TissueLyser from Qiagen, Germany has proven especially suitable. Other suitable devices include, for instance, Retsch Mixer mills, french press, mortar (seasand), Ultra Turrax, sonication, etc.

The bulk sample usually contains liquid, in particular water, and thus its consistency may be considered as being rather semi-solid than solid. In order to conveniently process such samples to the powder, it may be expedient to increase the consistency of the samples, e.g. solidify the samples.

Thus, according to a preferred embodiment, size reduction is carried out at reduced temperature and/or with prior drying. Temperatures below 0° C. and preferably below −20° C. are suitable. Reduced temperatures and/or drying may allow for a more efficient size reduction in case the sample is rather semi-solid than solid and/or minimize the decomposition of the analytes of interest. Reduced temperatures may be conveniently obtained by using dry ice and/or liquid nitrogen. Drying may be conveniently achieved by freeze-drying.

Depending on the organism(s) or part(s) thereof (bulk sample), size reduction may be carried out stepwise. This has the advantage that the means for size reduction can be selected more appropriately, depending on the particle size and consistency of the starting material and the particle size to be achieved. Accordingly, an intermediate material is obtained which results from at least one size reduction step and which is subjected to at least one further size reduction step. In practice, said intermediate material can be processed further directly after it is obtained, or stored for processing later on.

For instance, homogenizing the organism(s) or part(s) thereof may comprise a previous step of size reduction to convert the organism(s) or part(s) thereof (bulk sample) to a particulate material that can then be conveniently processed to a powder.

This previous step of size reduction is well known to those skilled in the art and may comprise, for instance, mixing, and/or cutting in any order, depending on the organism(s) or part(s) thereof.

Such a size reduction can be carried out manually or using suitable devices such as mixers or cutters. For plant material, cutting and mixing devices such as the Urschel Comitrol model 2600 food cutter, Stephan model 40 vertical cutter/mixer or Hobart HCM 450 vertical cutter/mixer may be used.

The resulting powder can then directly be used for the recording step or is first further converted into a form accessible for recording the spectra.

Further converting the powder into a form accessible for recording the spectra may, for instance, comprise the provision of a suspension of the powder. To this end, a liquid such as water may be added to the powder. The amount of liquid to be added to a given amount of powder can be easily determined by the skilled person. However, it should be chosen so that a relatively small volume of suspension can be used. For instance, 1 mg to 100 mg of powder in 100 μl to 1000 μl of liquid has proven to be suitable.

The provision of a powder suspension is intended to facilitate transfer of the sample to the recording device. Subsequent to the transfer the water may be removed again. This can be achieved by subjecting the suspension to conditions which allow the water to evaporate, for example by means of placing the suspension in an oven at elevated temperatures (up to 100° C.) and/or by applying vacuum.

Alternatively, the supernatant of the suspension can be used for recording. To this end, the powder suspension may be centrifuged and at least a part of the supernatant transferred to the recoding device. Subsequent to the transfer the water may be removed again and the spectrum recorded on the remaining residue. Said residue represents an extract of the organism(s) or part(s) thereof and the skilled artisan will readily appreciate that other liquids than water may be expedient for this purpose.

c) Recording

The analysis of the invention comprises recording at least one spectrum from the group of IR, FT-IR, Raman, FT-Raman and Near Infrared (NIR) and thus obtaining information about the organism at the time of testing.

It is a particular advantage of the method of the present invention that the amount of sample aliquot used for the measurement can be rather small. Said small amounts conveniently allow the recording of more than 1 sample in parallel. In particular 2 to 24, 2 to 96 or even multiples thereof can be subjected to the recording in parallel. For instance, 24 well-, 96 well-microtiter or even 384-well or 1536-well plates can be used conveniently as recipients for the sample aliquots. Likewise, silicon microplates designed for the HTS-extension from BrukerOptics (or similar instruments from other companies) can be used.

It is possible in principle to employ all methods known to be suitable for recording IR, FT-IR, Raman, FT-Raman or Near Infrared (NIR) spectra.

The recording of IR spectra is typically performed in the spectral range of the so-called medium infrared, from 500 to 4,000 cm⁻¹, although it can also be measured in the near infrared range from 4,000 to 10,000 cm⁻¹ or extended to include this range.

Any of the known spectroscopic measurement arrangements can be used for the recording of IR or Raman spectra, such as transmission/absorption, attenuated total reflection, direct or diffuse reflection or IR fibre-optic techniques. The preferred method is measurement by transmission/ absorption.

For example, a Bruker TENSOR 27 FT-IR spectrometer with an attached high throughput screening extension HTS-XT can be used (or similar instruments from other companies).

The samples may be solid or liquid. The measurement is best carried out with the aid of multi-cuvettes for the measurement of several samples or the use of microspectrometric techniques. These include FT-IR, NIR, Raman and FT-Raman microscopy or other processes of beam focussing. This allows the amount of samples to be reduced to a minimum and the use of an automated sample preparation and measurement procedure, in order to increase the sample throughput and establish a level for high-throughput screening. Sample carriers, as used for micro-titration plates, or throughflow cuvettes can also be used. The use of throughflow cuvettes, coupled with an automated sample delivery system, would also enable an increased sample throughput. Infrared fibre-optics can also be used for automation of the measurement process more independent of the location.

All water-insoluble optical materials commonly used in IR spectroscopy can be used as materials for cuvettes or sample carriers for the preparation variants described above, such as Si, Ge, ZnSe, CaF₂, BaF₂, although ZnSe has proven very suitable as a multisample element. Keyed metal plates or micro-metal grills are also suitable as sample holders, particularly if they are designed to the same scale as the micro-titration plates for a large number of samples, and as disposable materials.

The sample volume for the recording of IR spectra can be kept very small, and need only be a few μl (2-20 μl). Depending on the given conditions with or without beam focussing, substance quantities in the μg-ng range can be used. The diameter of the sample areas illuminated varies between 1-6 mm and 5-50 μm with micro-focussing.

In the case of Raman or Near Infrared (NIR) measurements, another possibility is measurement in a liquid sample, which can be carried out direct in the sample preparation vessels, e.g. micro-titration plates. This can offer a considerable time benefit coupled with a high degree of automation, since the processing times are reduced and sample preparation steps can be omitted. The optimum positioning of the Raman signal can be achieved by the use of confocal beam guidance, in order to eliminate interference signals and improve the signal-to-noise ratio. An arrangement of simultaneously used light sources or the corresponding replication of the stimulating beam and direction onto the sample for the Raman measurement, and the use of detectors (e.g. CCDs) arranged in parallel, can also significantly increase the sample throughput and the automation capability.

d) Comparison

It is possible with the measurement methods described above to assign to each investigated sample a particular pattern which characterizes the biochemical and/or metabolic state of the organism(s) or part(s) thereof from which the sample is derived. By comparison of the test spectrum/spectra with one or more spectra (reference spectra), divided into one, two or more classes, of the same organism treated with reference substances the test spectra may be allocated to one, two or more of the classes of reference spectra in the reference database.

In the preferred embodiment of the invention, the comparison is carried out by means of mathematical processes of spectral pattern recognition. Preferably, the reference spectra and/or test spectra are processed in such a way as to allow the automatic recognition of the characteristic spectral patterns.

The classification may be carried out by means of a pattern recognition system that can distinguish between two or more classes simultaneously. The class specific information of a spectral pattern may be stored in a classification model or by means of weights in an artificial neural network, a support vector machine or equivalents.

The comparison of the test spectra with the reference spectra may be carried out by means of the classification model.

Due to their large number of components, these spectra have a very complex composition, and reflect many different vibration modes of the organism's biomolecules. Despite their complexity, the spectra are very specific of the composition, properties or condition of the organism and represent a specific, biochemical fingerprint. Since the composition, condition and properties of organism is likely to change in a specific way under the effect of treatment with a test substance, depending on the substance used, the spectroscopic recording of these changes can be used for the identification and/or characterisation of the action mechanism involved.

The method of the invention is particularly aimed at assessing the mode of action of a tested substance or substance mixture (herbicides, insecticides, fungicides, acaricides, and nematodicides). The properties to be assessed in particular include assessing the mode of action of substances (enzyme inhibitors, receptor agonists and antagonists, channel blocker, etc) which cause insecticidal effects, fungicidal effects, phytotoxic effects on plants (herbicides) and of substances which improve the health of a plant.

Mode of actions of insecticides to be assessed include in particular the following:

Acetylcholine esterase inhibition, GABA-gated chloride channel antagonism, sodium channel modulation, nicotinic acetylcholine receptor agonism or antagonism, chloride channel activation, juvenile hormone mimicing, inhibition of oxidative phosphorylation, disruption of ATP formation, uncoupling of oxidative phosphorylation via disruption of proton gradient, inhibition of chitin biosynthesis, moulting disruption, ecdysone agonism, octopaminergic agonism, mitochondrial complex III electron transport inhibition, voltage-dependent sodium channel blocking, inhibition of lipid synthesis, mitochondrial complex IV electron transport inhibition, neuronal inhibition, aconitase inhibition, P450-dependent monooxygenase inhibition, esterase inhibition, ryanodine receptor modulation.

Mode of actions of fungicides to be assessed relate in particular to one of following:

Nucleic acids synthesis, target sites including RNA polymerase I, adenosine deaminase, DNA/RNA synthesis, and DNA topoisomerase type II (gyrase); mitosis and cell division, target sites including β-tubuline assembly in mitosis, β-tubulin assembly in mitosis, and cell division; respiration, target sites including complex I, complex II: succinate dehydrogenase, complex III: cytochrome bc1 (ubiquinol oxidase) at Qo site, complex III: cytochrome bc1 (ubiquinone reductase) at Qi site, uncoupler of oxidative phosphorylation, inhibitors of oxidative phosphorylation, ATP synthase, and ATP production; amino acids and protein synthesis, target sites including methionine biosynthesis, and protein synthesis; signal transduction, target sites including G-proteins in early cell signalling, and MAP protein kinase in osmotic signal transduction; lipids and membrane synthesis, target sites including NADH cytochrome c reductase in lipid peroxidation, phospholipid biosynthesis, methyltransferase, lipid peroxidation, cell membrane permeability, and phospholipid biosynthesis and cell wall deposition; sterol biosynthesis in membranes, target sites including C14-demethylase in sterol biosynthesis, Δ14-reductase and Δ8→Δ7-isomerase in sterol biosynthesis, 3-keto reductase, C4-demethylation, and squaleneepoxidase in sterol biosynthesis; glucan synthesis, target sites including trehalase and inositolbiosynthesis, and chitin synthase; melanin synthesis in cell wall, target sites including reductase in melanin biosynthesis, and dehydratase in melanin biosynthesis; host plant defence induction, target sites including salicylic acid pathway.

Mode of actions of herbicides to be assessed include in particular the following:

Inhibition of acetyl CoA carboxylase (ACCase), inhibition of acetolactate synthase ALS (acetohydroxyacid synthase AHAS), inhibition of photosynthesis at photosystem II, photosystem-I-electron diversion, inhibition of protoporphyrinogen oxidase (PPO), bleaching (inhibition of carotenoid biosynthesis, e.g., at the phytoene desaturase step (PDS), inhibition of 4-hydroxyphenyl-pyruvate-dioxygenase (4-HPPD)), inhibition of EPSP synthase, inhibition of glutamine synthetase, inhibition of DHP (dihydropteroate) synthase, microtubule assembly inhibition, inhibition of mitosis and/or microtubule organisation, Inhibition of VLCFAs, inhibition of cell wall (cellulose) synthesis, uncoupling (membrane disruption), inhibition of lipid synthesis, action like indole acetic acid, inhibition of auxin transport.

The modes of action referred to are examples only, and are by no means exhaustive, and more could easily be added by any specialist in the field.

The reference database is built up by exposing an organism or a group of organisms to a substance or substance mixture whose mode of action is known, optionally converting the organism or group of organisms or part(s) thereof into a homogeneous sample, and recording at least one reference spectrum. The conditions of exposure and conversion define the conditions under which the same organism or group of organisms or part(s) thereof is exposed to a test substance or a mixure of test substances whose mode of action is unknown and optionally converted into a homogeneous sample. The reference spectra are added to the database, allocated to the class belonging to the relevant mode of action.

The reference spectra allocated to a class show an identical or similar structure in one or more of the selected wavelength ranges, which differs significantly from the structure of the reference spectra of other classes in the selected wavelength ranges.

The selection of the wavelength ranges used for the differentiation of the classes (”feature selection”) can be made by means of multi-variate statistical procedures, such as variance analysis, co-variance analysis, and factor analysis, statistical distance dimensions such as the Euclidian distance or the Mahalanobis distance, or a combination of these methods together with an optimisation process such as genetic algorithms.

An automated and optimised search for wavelengths can be performed through the use or combination of genetic algorithms. In this way, the wavelengths can be compiled into a ranking more quickly and efficiently, in the best way possible for the classification. The main feature here is that an automated identification is performed of the spectral changes which make a contribution to the spectral change. These identified ranges can be used in order to build up an automated classification system. The evaluation is ideally made through a combination of genetic algorithms with the co-variance analysis.

Prior to the wavelength selection, preliminary processing of the reference spectra can be carried out in order to increase the spectral contrast by means of the formation of derivations, normalization, deconvolution, filtering, noise suppression or data reduction by wavelet transformation or factor analysis.

The allocation of the reference spectra into the different classes is carried out by means of mathematical classification methods such as multi-variate, statistical pattern recognition, neuronal networks, support vector machines, linear discriminant analyses, methods of case-based classification or machine learning, genetic algorithms or methods of evolutionary programming. Several synthetic neuronal networks can be used as a feed-forward network with three layers and a gradient decent method as the learning algorithm. The classification system may show a tree structure, in which classification tasks are broken down into partial tasks, and the individual classification systems in a unit are combined to form a hierarchical classification system, in which all stages of the hierarchy are processed automatically during the course of the evaluation. The individual stages of the classification systems may take the form of neuronal networks, which have been optimised for special tasks.

A combination of neuronal networks with a genetic algorithm is also possible to undertake an optimisation of the classification through neuronal networks. This optimisation can for example be carried out by improvement of the network architecture or the learning algorithm.

The reference database can also take the form of a synthetic neuronal network, in which the spectral information is stored in the form of neuronal weights, and can be sued in the evaluation.

The creation of the reference database for the characterisation and/or identification of the modes of action in an organism in principle need be carried out only once. There also exists the facility of extending the database at any time. This can be done, for example, by adding further substances to the classes already contained in the database. Apart from this, the reference database can also be extended to include other modes of action not so far contained in the database. In such cases, the database must be reorganised as described above, whereby the spectral data records already used for the creation of the previous database do not need to be re-created as long as the organism used, the conditions of exposure and sample preparation, and the spectral measurement parameters are not changed.

The allocation of a test spectrum to one, two or more classes of reference spectra can be made by means of mathematical classification methods based on pattern recognition. Methods that enable simultaneous classification into several classes, such as is the case with classification by means of synthetic neuronal networks, are particularly suitable for the automated and efficient classification of several classes. Processes based on the probability density function, the correlation matrix, methods of case-based classification or machine learning, genetic algorithms or methods of evolutionary programming are also suitable in principle. The classification system may consist of several sub-units with a tree structure, in which classification tasks are broken down into partial tasks, and the individual classification systems in a unit are combined to form a hierarchical classification system, in which all stages of the hierarchy are processed automatically during the course of the evaluation.

The test spectrum of a substance or substance mixture with an unknown mode of action is obtained with exactly the same type of organism or group of organisms or part(s) thereof that is or has been used for the recording of the reference spetra. All conditions which might influence the biochemical and/or metabolic state of the organism or group of organisms or part(s) thereof must also correspond to those maintained during the creation of the reference database.

The allocation of a test spectrum to one, two or more classes of reference spectra is carried out by means of mathematical classification methods such as multi-variate, statistical processes of pattern recognition, neuronal networks, methods of case-based classification or machine learning, genetic algorithms or methods of evolutionary programming.

According to a particular embodiment, the present invention relates to a method for the identification and/or characterisation of the mode of action of a substance or substance mixture, in particular a pesticide or a mixture of pesticides, wherein the method comprises the following steps:

compilation of reference spectra by means of the treatment of an organism or a group of organisms or part(s) thereof with substances or substance mixtures, in particualar pesticides, whose mode of action is known, and recording of at least one spectrum from the group of IR, FT-IR, Raman, FT-Raman and Near Infrared (NIR) spectra; in each case, selection of at least one wavelength range of the same or similar structure to differentiate between the classes belonging to the corresponding mode of action, and allocation of the reference spectra into the classes in the reference database, whereby the reference spectra allocated to a class demonstrate an identical or similar structure in the selected wavelength range, which differs significantly from the structure of the reference spectra of other classes in the selected wavelength range; treatment of an organism or a group of organisms or part(s) thereof with the substance or substance mixture, in particular the pesticide or pesticide mixture, to be tested; recording of at least one spectrum (test spectrum) from the group of IR, FT-IR, Raman, FT-Raman and Near Infrared (NIR) spectra; comparison of the test spectrum/spectra from with one or more reference spectra in the reference database; allocation of the test spectra to one, two or more classes of reference spectra in the reference database and identification or characterisation of the mode of action.

The method of the invention is characterised by the fact that it is sensitive, can be standardised and is reproducible. It is generally and uniformly applicable to the most varying action mechanisms. It is cost-effective and provides quick results.

The present invention therefore relates further to the use of the method of the invention for the aforementioned purposes. This is connected in particular with the analytical finding of whether exposure to a substance or to a substance mixture leads to a change in the organism that is representative for a certain mode of action. If this is so, the mode of action is allocated to the substance or the substance mixture.

### DESCRIPTION OF THE FIGURES

In the drawings

FIG. 1 shows a 12-well microtiter plate with aphids;

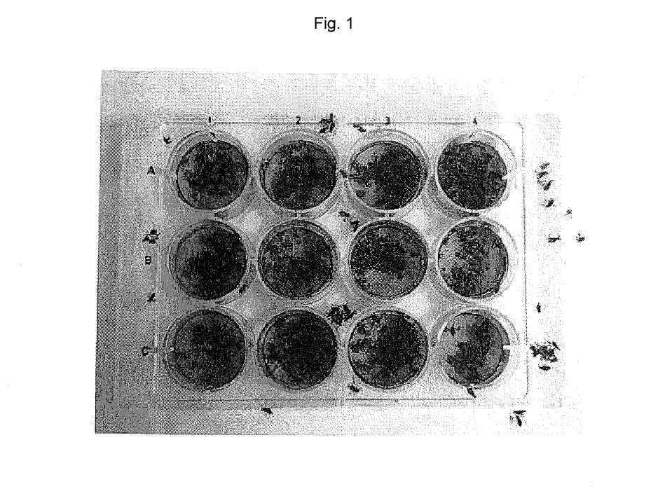

FIG. 2 shows a Teflon block for collection of the aphids attached to the microplate;

FIG. 3 shows a tube holder for freeze-drying procedure;

FIG. 4 shows (A) a tube containing freeze-dried aphids and (B) a tube containing freeze-dried aphids after bead milling;

FIG. 5 shows a 96-”well” silicon microplate with 12 different aphid samples (columns), each in seven repeats (rows);

FIG. 6 shows the result of a hierarchical clustering (Ward's algorithm) of the IR spectra from an experiment, where insects (vetch aphids) were incubated with different substances;

FIG. 7 shows the result of a hierarchical clustering (Ward's algorithm) of the IR spectra from an experiment, where whole plants (Lemna) were incubated with different substances;

FIG. 8 shows the result of a hierarchical clustering (Ward's algorithm) of the IR spectra from an experiment, where plant pathogenic fungi (pyricularia oryzae) were incubated with different substances.

### EXAMPLES

1 Insects (Megoura viciae): Testing Insecticides for their Mode of Action

### 1.1 Preparation of Microtiterplates

Each well of a 12-well microtiterplate (supplier: TPP, wells: φ 22.2 mm) was filled with 1.4 ml of 0.8% Agar-Agar containing 2.5 ppm of the fungicide Opus to avoid fungal infections of the Agar-Agar.

After cooling a 20 mm broad bean (viciae faba) leaf disk was placed on the agar (bottom side up).

### 1.2 Application of Substance

Ultrasonic-spraying with concentrations between 0.3 ppm and 2500 ppm of the corresponding substances was used to treat the leaf discs. The standard volume application rate was 20 μl with different concentrations of substances dissolved in a 1:1 acetone/water mixture.

For the negative controls leaf discs were treated with a 1:1 acetone/water mixture.

### 1.3 Drying of the Leaf Disks

The sprayed microtiterplates were either stored under a fume hood for a quick dry (app. 2-4 hours) or overnight in a climatized test chamber at 23±1° C., 50±5% RH and 3500±500 lux of fluorescent bulb light (app. 12 hours).

### 1.4 Infection

After the drying of the leaf disks approx. 30 aphids (Megoura viciae) were placed on each leaf disk.

### 1.5 Incubation

The top side of the microtiterplates was covered by cellulose and the associated lids. In order to prevent the wells from too much moisture each lid had twelve 4 mm-holes drilled centric atop the wells.

The treated microtiterplates were incubated for 24-48 hours in a climatized test chamber at 23±1° C., 50±5% RH and 3500±500 lux of fluorescent bulb light.

### 1.6. Sample Preparation

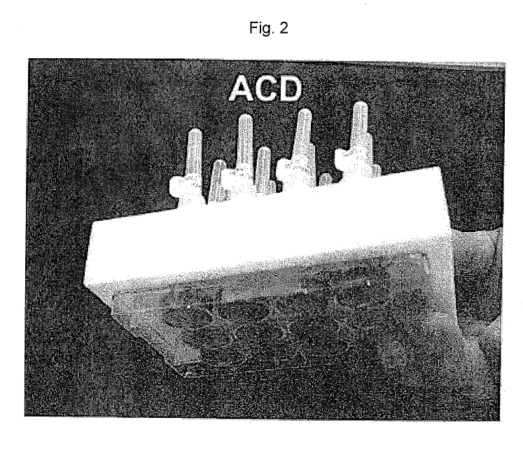

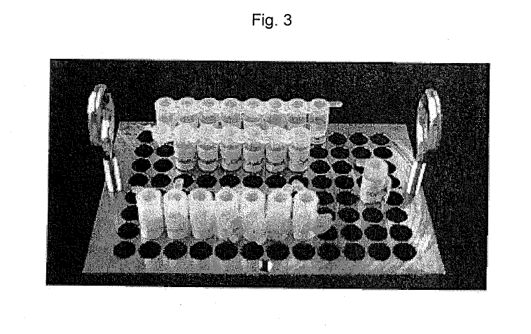

Insects were collected using a home-made teflon-block with 12 millcutted funnels. On the bottom of each funnel a collection mictrotube was mounted by means of a small piece of silicon tube. The whole block (=aphid collection device, ACD) was then attached to the microplate (see FIG. 2). The ensemble was flipped and aphids were transferred into the collection tubes. The tubes were closed with perforated caps, placed in a holder fabricated from brass (FIG. 3), cooled down in liquid nitrogen and freeze-dried for one day (Freeze Dryer Christ ALPHA 2-4 approx. 0.024 mbar). A steel ball was added to each tube. After closing the tubes (this time using an intact cap) the aphids were ground for 2 min with 25 Hz in a bead mill (TissueLyser from Qiagen) processing up to 96 tubes in parallel. The resulting powder was resuspended in the microtubes after the addition of 300 μl Baker water.

### 1.7. IR Measurement

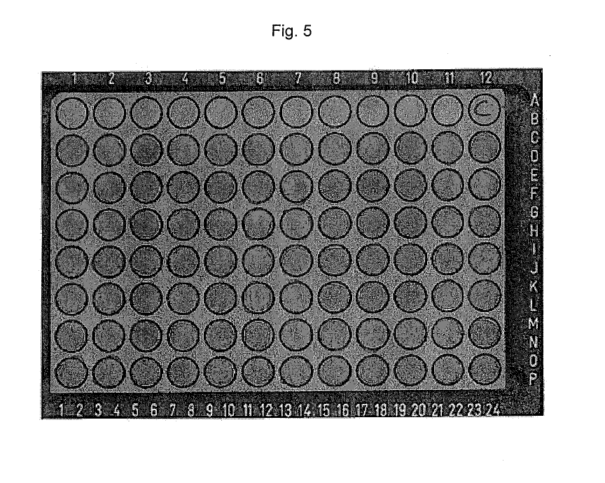

10 μl from the suspension above were transferred to a measuring position of a silicon 96-“well”plate (instead of the whole suspension it was also possible to use only the supernatant as sample for the IR measurements). By means of the pipette tip the samples were distributed within the whole area of the corresponding imprinted ring. Each sample was such applied to seven different positions, 12-13 samples could be processed on one single plate (see FIG. 5). The silicon plate was dried at 60° C. for 60 minutes, equilibrated to room temperature for 5 minutes and immediately subjected to measurement.

The infrared spectra were recorded in transmission on a Bruker TENSOR 27 FT-IR spectrometer with an attached high throughput screening extension HTS-XT. The spectrometer was controlled by the OPUS software package from BrukerOptics. The following parameters were used to record the FT-IR spectra: spectroscopic region: 4000-400 cm⁻¹, resolution 8 cm⁻¹, 16 scans per well, aperture 6 mm, mirror speed 10 kHz.

### 1.8. Data Analysis

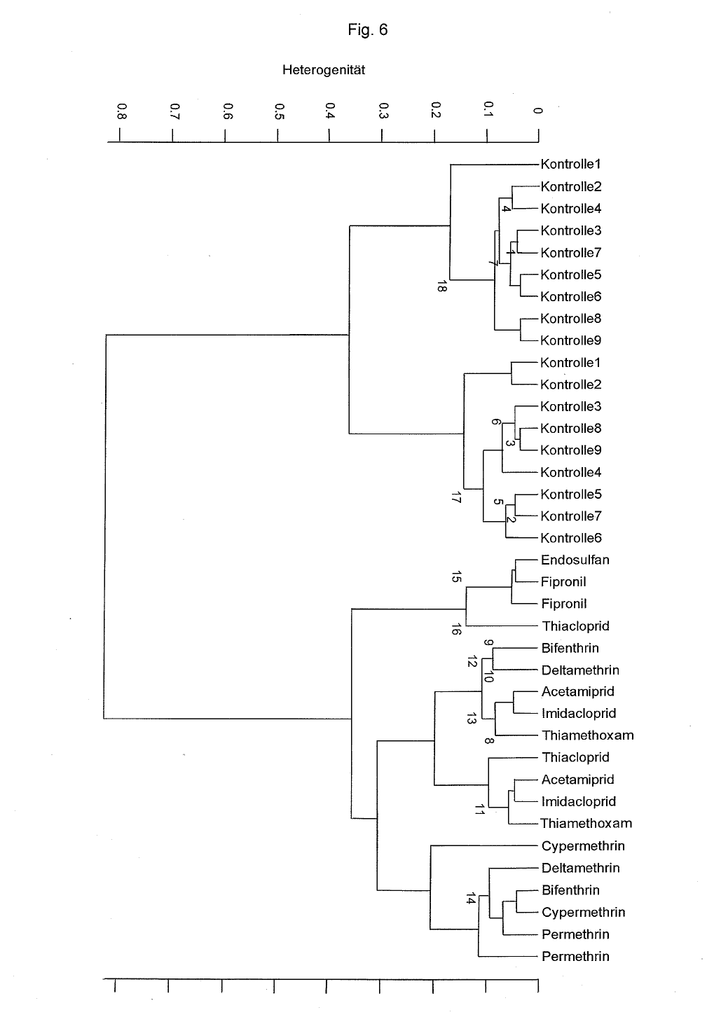

With the IR spectra a simple hierarchical clustering using Ward's algorithm was performed. The resulting dendrogram (FIG. 6) showed two major branches. All control samples showed up in one branch, while the treated samples completely concentrate in the second branch. The treated samples were again split up mainly into three groups. With only a few exceptions this three groups could be assigned as group a), comprising the compounds acting on the nicotinic acetylcholine receptor (thiamethoxam, acetamiprid, thiacloprid and imidacloprid), group b), including most of the sodium channel modulators (bifenthrin, cypermethrin, deltamethrin, and permethrin), and group c), representing mainly the GABA-gated chloride channel antagonists (endosulfan and fipronil).

2) Plants (Lemna Paucicostata): Testing Herbicides for their Mode of Action

For the Lemna test, stock cultures of Lemna paucicostata (L.) Hegelm. (collection Prof. R. Kandeler, University of Vienna, Austria) were propagated mixotrophically in an inorganic medium containing sucrose (K. Grossmann, Pest Management Science 61: 423-431, 2005). The bioassay was conducted under aseptic conditions in plastic Petri dishes (5 cm in diameter, six replicates) which contained 15 ml medium without sucrose. The test compounds were added to the dishes in acetone solution at concentrations of 10⁻⁵M and 10⁻⁶M, and the organic solvent allowed to volatize before loading them with ca. 120 fronds each.

The following compounds were applied: Topramezone (inhibitor of carotenoid synthesis), picolinafen (inhibitor of carotenoid synthesis), diuron (inhibitor of photosystem II), and chlorsulfuron (inhibitor of acetolactate synthase). Controls received corresponding amounts of acetone alone.

The culture dishes were then closed with plastic lids and incubated under continuous light (Philips T L white neon tubes, 40 μmol m⁻² s⁻¹ photon irradiance, 400 to 750 nm) in a growth chamber at 25° C. After 48 hours of treatment with cornpounds, plants from three replicate dishes were sampled (200 mg total fresh weight) in microtubes. The microtubes were placed in a holder fabricated from brass (compare FIG. 3) and freeze-dried. After the additions of one steel ball to each tube the lemna were ground in a bead mill processing 96 tubes in parallel. The resulting powder was resuspended in water and transferred to a 96-“well” silicon plate. After drying of the plate the FT-IR spectra were recorded.

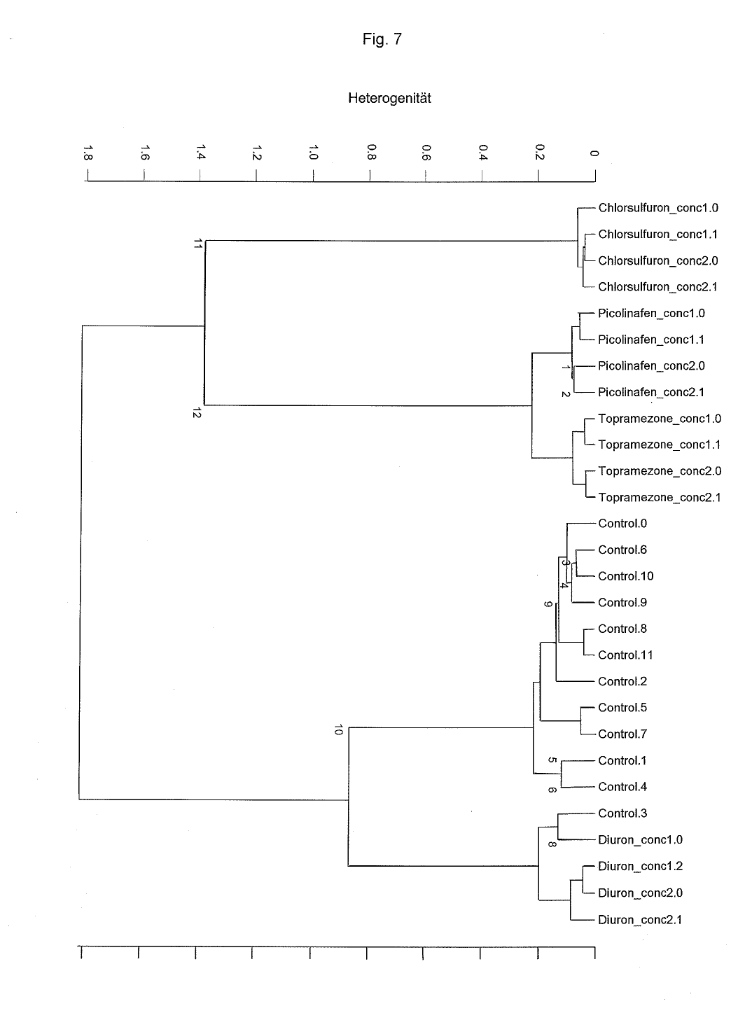

The resulting dendrogram (FIG. 7, hierarchical clustering using Ward's algorithm) showed that IR analysis assigned spectral changes induced by the carotenoid synthesis inhibitor topramezone to those of picolinafen which also inhibits carotenoid synthesis.

In contrast, the dendrogram also revealed clear separation of spectral changes by the carotenoid synthesis inhibitors to those induced by compounds with different mode of action such as the photosystem II inhibitor diuron and the acetolactat synthase inhibitor chlorsulfuron.

3) Fungi (Pyricularia Oryzae): Testing Fungicides for their Mode of Action

Stock cultures of the phytopathogenic fungus Pyricularia oryzae were plated on agar plates and incubated at room temperature. After two weeks spores were harvested from the agar by suspending in malt medium and filtered. The spores were diluted to 25 ml and incubated in a 100 ml flask at room temperature and shaking (140 rpm). After 24 h starting cultures were combined, homogenized and redistributed in 100 ml flask. After 5 days test compounds were added and incubation continued for 24-48 h. Harvest was conducted by suction on filter paper in buechner funnels.

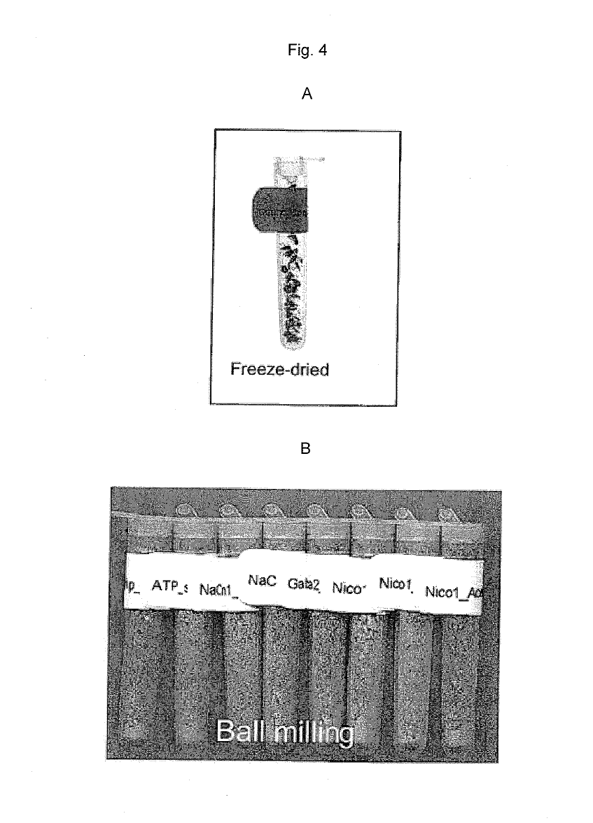

The mycellium was transferred into microtubes, which then were placed in a holder fabricated from brass (compare FIG. 3) and freeze-dried. After the additions of one steel ball to each tube the fungi were ground in a bead mill (compare FIG. 4B) processing 96 tubes in parallel. The resulting powder was resuspended in water and transferred to a 96-“well” silicon plate (compare FIG. 5). After drying of the plate the FT-IR spectra were recorded.

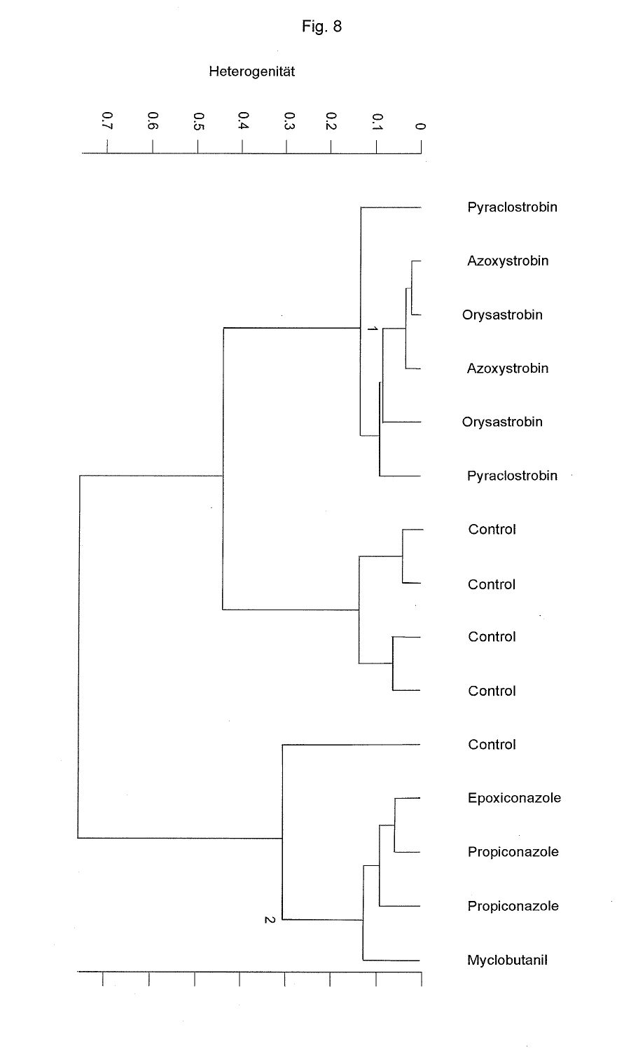

A hierarchical clustering using Ward's algorithm was performed with the IR spectra. The resulting dendrogram (FIG. 8) showed three major branches. Most of the controls showed all up in one branch, while all compounds acting on the bc₁-complex (pyraclostrobin, azoxystrobin, and orysastrobin) clustered together in the second branch. The third branch of the dendrogram was mainly formed by the inhibitors of the C14-demethylase in the sterol biosynthesis (epoxiconazole, propioconazole, myclobutanil).

1-10. (canceled)

11. A method for identifying and/or characterizing the mode of action of a test substance or test substance mixture, which comprises:
a) exposing an organism or a group of organisms to the test substance or to the test substance mixture;
b) optionally converting the organism or group of organisms or part(s) thereof into a homogeneous sample;
c) recording at least one test spectrum from the group of IR, FT-IR, Raman, FT-Raman and Near Infrared (NIR) on the organism, group of organisms, or sample; and
d) comparing the at least one test spectrum with a database comprising reference spectra, wherein the reference spectra are allocated to classes of reference spectra, and allocating the at least one test spectrum to at least one class of reference spectra thereby identifying and/or characterizing the mode of action of the substance or substance mixture.

12. A method for determining the biochemical and/or metabolic state of an organism or a group of organisms or part(s) thereof, which comprises:
a) exposing the organism or the group of organisms to a test substance or to a test substance mixture;
b) optionally converting the organism or group of organisms or part(s) thereof into a homogeneous sample;
c) recording at least one test spectrum from the group of IR, FT-IR, Raman, FT-Raman and Near Infrared (NIR) on the organism, group of organisms, or sample; and
d) comparing the at least one test spectrum with a database comprising reference spectra, wherein the reference spectra are allocated to classes of reference spectra, and allocating the at least one test spectrum to at least one class of reference spectra thereby determining the biochemical and/or metabolic state of the organism or a group of organisms or part(s) thereof.

13. The method according to claim 11, wherein the organism is selected from arthropods, nematodes and mollusks.

14. The method according to claim 13, wherein the organism is Megoura viciae.

15. The method according to claim 11, wherein the organism is a plant.

16. The method according to claim 15, wherein the plant is Lemna.

17. The method according to claim 11, wherein the organism is selected from phytopathogenic fungi.

18. The method according to claim 11, which comprises step b).

19. The method according to claim 18, wherein converting the organism or group of organisms or part(s) thereof into a homogeneous sample comprises homogenizing the organism or group of organisms or part(s) thereof.

20. The method according to claim 19, wherein homogenizing the organism or group of organisms or part(s) thereof comprises subjecting the organism or group of organisms or part(s) thereof to size reduction.

21. The method according to claim 12, wherein the organism is selected from arthropods, nematodes and mollusks.

22. The method according to claim 21, wherein the organism is Megoura viciae.

23. The method according to claim 12, wherein the organism is a plant.

24. The method according to claim 23, wherein the plant is Lemna.

25. The method according to claim 12, wherein the organism is selected from phytopathogenic fungi.

26. The method according to claim 12, which comprises step b).

27. The method according to claim 26, wherein converting the organism or group of organisms or part(s) thereof into a homogeneous sample comprises homogenizing the organism or group of organisms or part(s) thereof.

28. The method according to claim 27, wherein homogenizing the organism or group of organisms or part(s) thereof comprises subjecting the organism or group of organisms or part(s) thereof to size reduction.
